(19) United States
(12) Patent Application Publication
(10) Pub. No.: US 2024/0052022 A1
(43) Pub. Date: Feb. 15, 2024

(54) ENGINEERED PICOBODY TO ALPHA SYNUCLEIN

(71) Applicant: Qatar Foundation for Education, Science and Community Development, Doha (QA)

(72) Inventors: Prasanna R. Kolatkar, Doha (QA); Zeyaul Islam, Doha (QA); Issam Hmila, Doha (QA); Nishant Vaikath, Doha (QA); Omar El-Agnaf, Doha (QA)

(21) Appl. No.: 18/233,733

(22) Filed: Aug. 14, 2023

Related U.S. Application Data
(60) Provisional application No. 63/398,113, filed on Aug. 15, 2022.

(51) Int. Cl. C07K 16/18; G01N 33/68

(52) U.S. Cl. CPC C07K 16/18; G01N 33/6896; C07K 2317/569; G01N 2800/2821; C07K 2317/76; C07K 2317/34; G01N 2800/2835

(57) ABSTRACT

Alpha synuclein picobodies, which are N-terminal truncations of alpha synuclein nanobodies are provided. Methods of using alpha synuclein picobodies for diagnosing and treating synucleinopathies are also provided.

### CROSS REFERENCE TO RELATED APPLICATIONS

This application claims the benefit of U.S. Provisional patent application 63/398,113 filed Aug. 15, 2022, the entire contents of which is incorporated by reference herein.

### FIELD

The present disclosure relates to compounds and methods for diagnosis and treatment of synucleinopathies.

### BACKGROUND

Nanobodies have become a useful tool for both diagnostics and potential therapeutics. Unlike conventional antibodies, nanobodies are composed of a single monomeric variable antibody domain. Thus, their molecular weight is ˜12-15 kDa versus antibodies having a molecular weight of ˜150-160 kDa as antibodies are composed of two heavy and two light protein chains. Originally, nanobodies were engineered from camelid heavy-chain antibodies but phage panning was subsequently used to produce large amounts in bacteria cells. The smaller size makes them easier to produce and potentially makes them more bioavailable for cells.

There is a need to be able to take advantages of these differences to be able diagnose and treat synucleinopathies.

### SUMMARY

The present disclosure provides picobodies, as well as compositions and methods of use thereof. Also disclosed are embodiments for diagnosing, differentiating, and treating patients with synucleinopathies. A picobody is a nanobody that is shorter in size by having, for example, 8 N-terminal amino acid residues removed.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows the structure of alpha synuclein peptide bound nanobody as a “cartoon” as well as surface representation of the overall architecture of the nanobody bound alpha synuclein peptide complex. In the surface representation, the nanobody is shown as [symbol] , the CDR regions as [symbol] , and the alpha synuclein peptide is shown as [symbol] .

FIG. 2 depicts nanobody-alpha synuclein peptide binding pocket: 2Fo-Fc electron density map of the α-syn peptide contoured at 1.0 σ. Alpha synuclein peptide is shown as < X, and the nanobody is shown as [symbol] . The amino acid residues involved in interaction between alpha synuclein peptide and the nanobody are shown as stick.

FIGS. 3A-3B depict alanine scanning carried out by two methods. FIG. 3A is by coating the peptides onto an ELISA plate and probing with the Nb-01; and FIG. 3B is by preincubating the peptides with Nb-01 followed by adding the complex to ELISA plate coated with alpha synuclein. Peptides used: 43-56 (14 aa peptide that was identified as the region recognized using the peptide library) and the same peptide with amino acid replaced with alanine from 46 to 52. Using indirect ELISA by coating the peptides, reduced signal was observed when amino acids 48 until 52 were replaced with alanine suggesting it is a part of the epitope. This was also confirmed by a preabsorption experiment in which a high signal was observed when amino acids 48-52 were replaced.

FIG. 4 depicts comparison to apo Nanobody (PDBID: 6APO) and comparison to apo Nanobody (PBDID: 5N LW).

FIG. 5 shows comparison with Nb in complex with C-terminal peptide of alpha synuclein (PDBID: 2X6M).

FIG. 6 shows comparison to SLHR: The catalytic domain of murine urokinase-type plasminogen activator in complex with the active site binding inhibitory nanobody Nb22 and comparison to 6I8H: Structure of the plant immune signaling node EDS1 (enhanced disease susceptibility 1) in complex with nanobody ENB15.

FIG. 7 shows the effect of NbM01 and DeltaNbM01 on the seeded aggregation assay of alpha synuclein. (monomers alpha synuclein 25 μM and 3 μM of seeds were used. Two ratio of Nbs (12 μM and 18 μM) were tested. The control represents a mixture of only monomers α-syn at 25 μM and seeds at 3 μM.

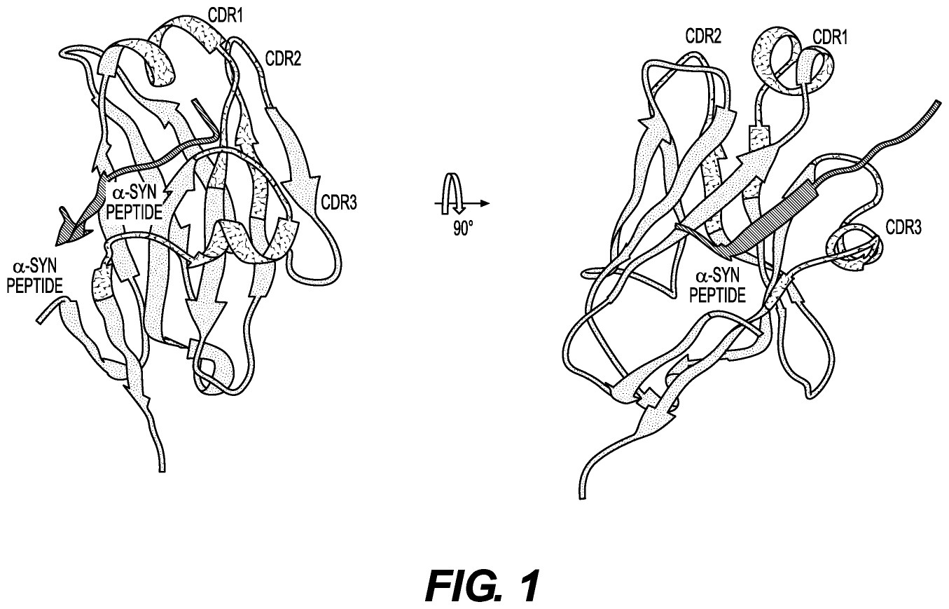

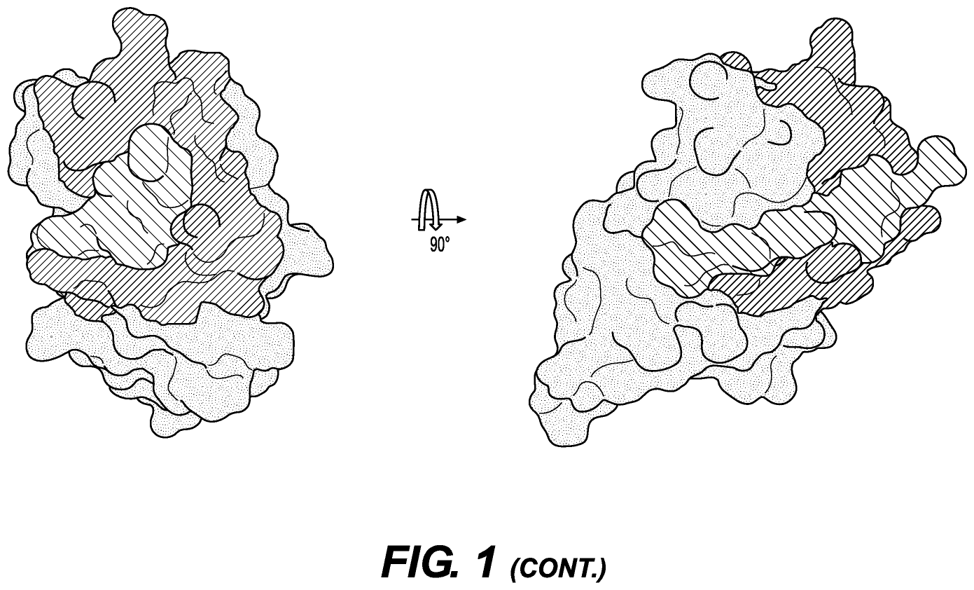

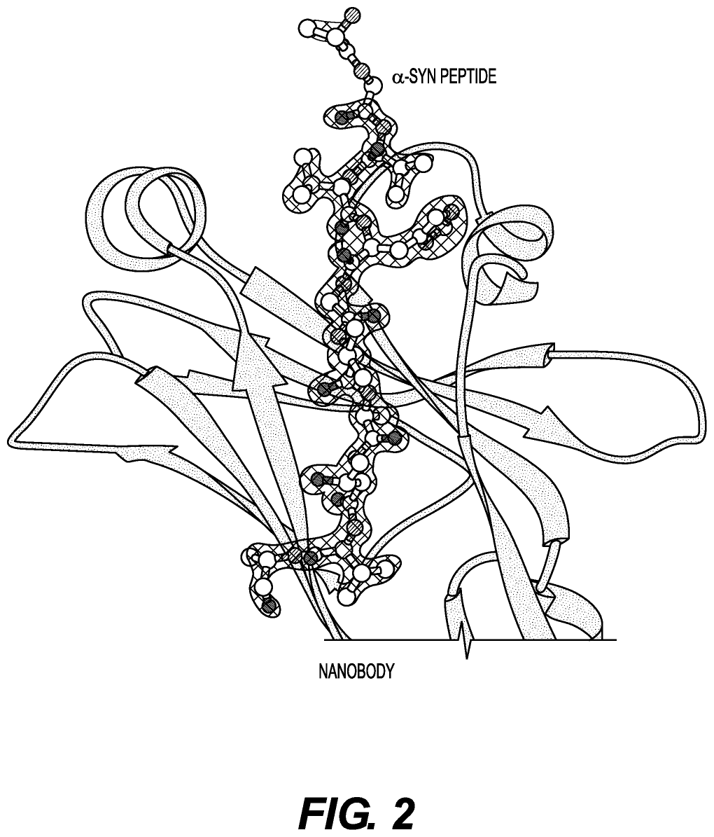

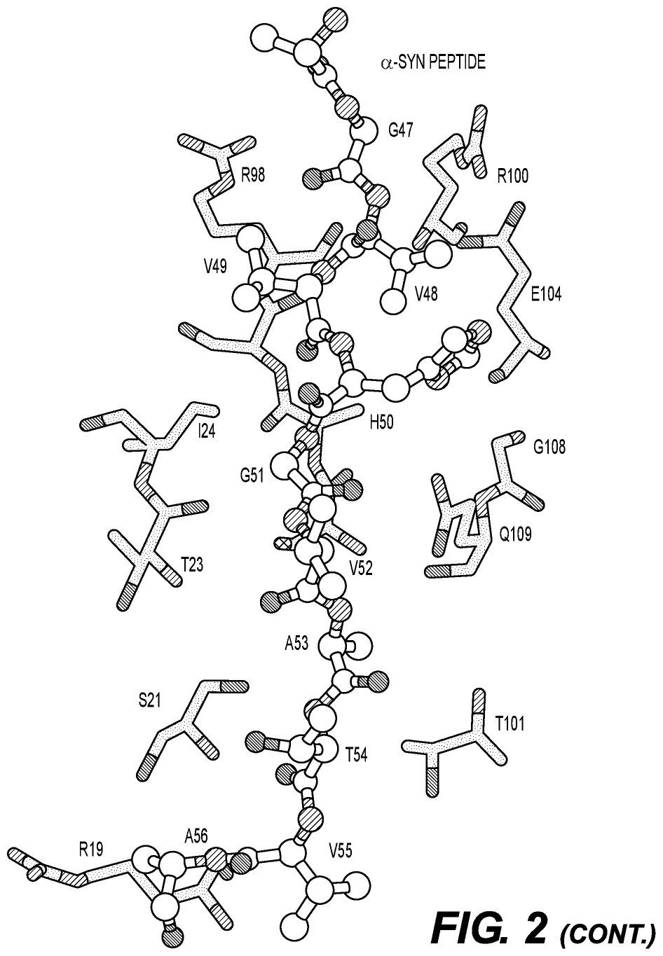

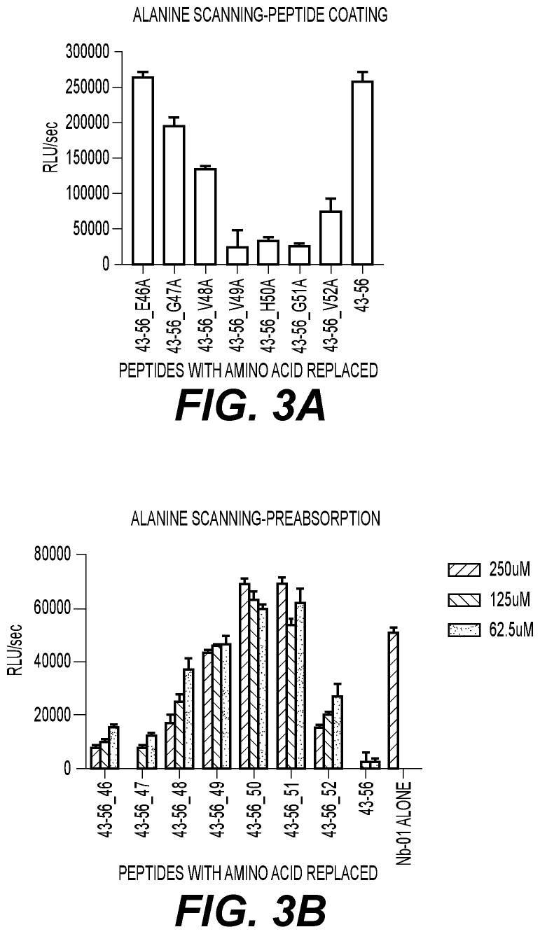

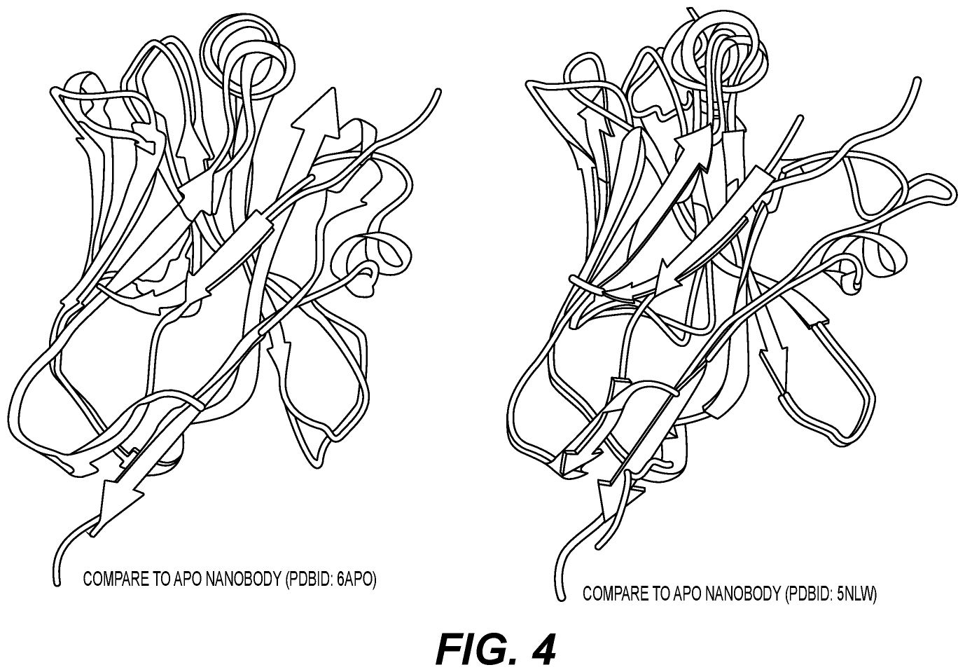

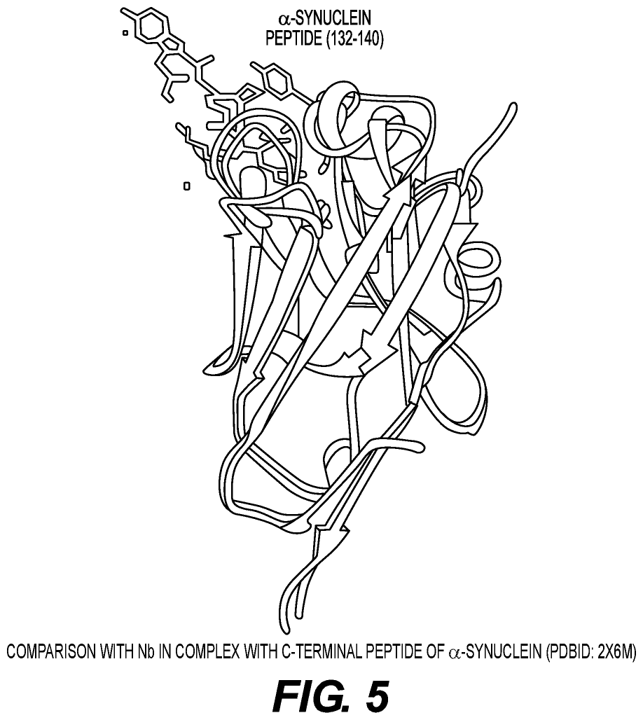

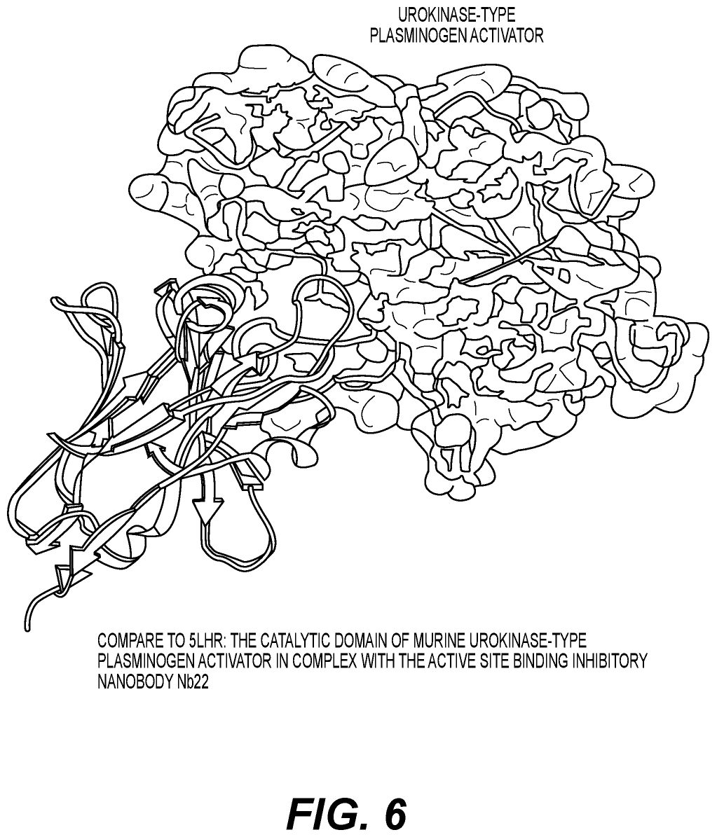

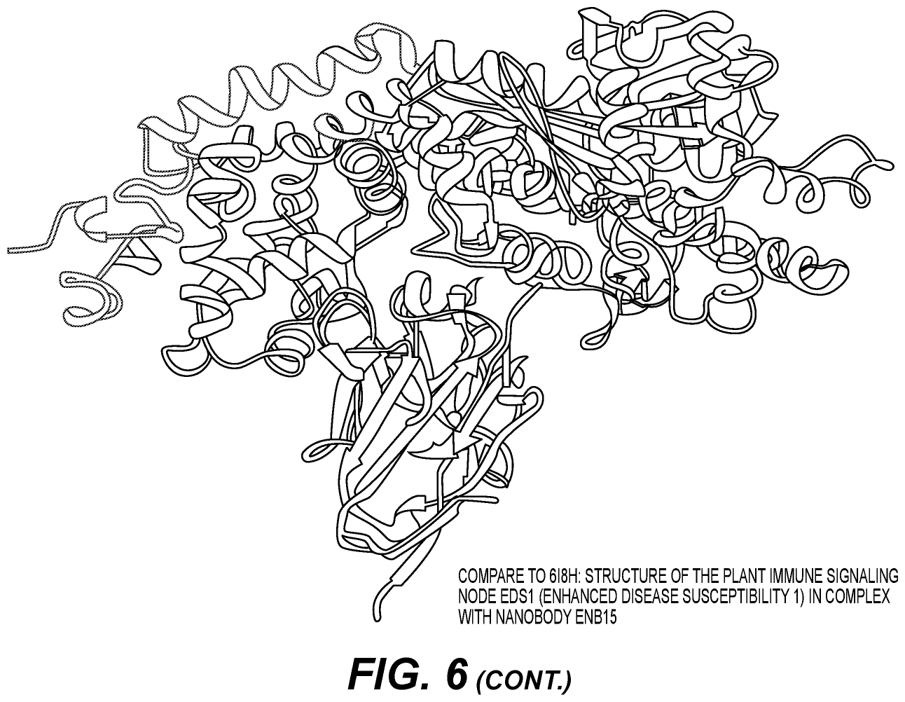

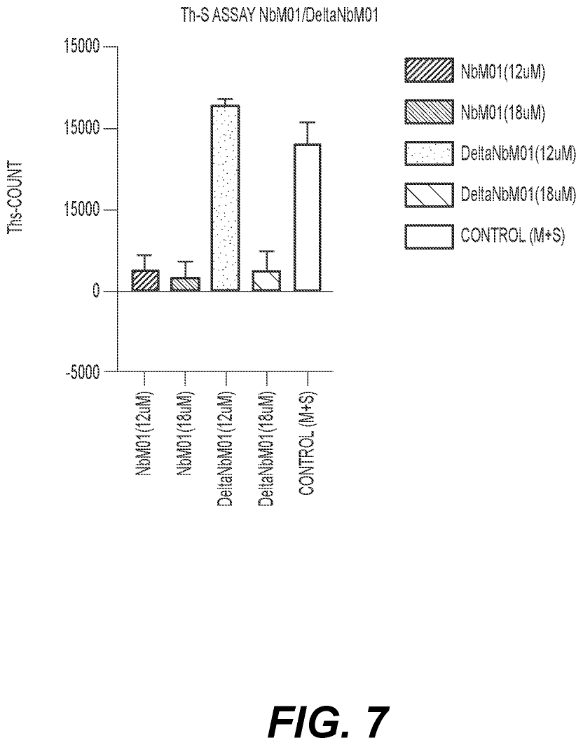

### DETAILED DESCRIPTION

Alpha synuclein protein is a core pathological feature of Parkinson's Disease (PD) and dementia with Lewy bodies (DLB). One potential mechanism of how alpha synuclein pathology spreads in PD and DLB is by template-directed aggregation of alpha synuclein.

Alpha synuclein has been targeted by several nanobodies for both diagnostic and therapeutic purposes. Using structural information resulting from X-ray crystallographic analysis, a shorter form of conventional nanobodies, referred to as a “picobody” was engineered. The newly designed picobody comprises a truncated N-terminal (8 residues) and still retains the ability to bind alpha synuclein and inhibit its aggregation. The smaller lattice makes it potentially more likely to have increased bioavailability and to better cross the brain-blood barrier.

Antibodies and nanobodies have been used for a diverse set of applications including diagnostics and therapeutics for decades. These protein molecules have high specificity and affinity for their respective targets. Recently, the El-Agnaf laboratory at QBRI developed and studied a nanobody targeting alpha synuclein (NbM01) which has high affinity to both monomers and oligomers of alpha synuclein. This nanobody was shown to bind and also prevent aggregation of alpha synuclein thus making it a diagnostic and potentially therapeutic nanobody.

The Kolatkar laboratory subsequently crystallized a NbM01-alpha synuclein complex and studied its three-dimensional structure using X-ray crystallography. Using a combination of home source and synchrotron X-rays (Brookhaven Lab), the Kolatkar group obtained a high resolution (1.25 angstrom) structure which allowed them to determine atomic level detail of the complex.

The complex revealed that the alpha synuclein is bound to a portion of the nanobody which previous proteins or peptides didn't bind to. This portion was away from the central portion of the complementarity-determining region 3 (CDR3) relative to previously determined structures and in addition, the alpha synuclein peptide displaced the N-terminal end of the nanobody. Alanine scanning of the alpha synuclein peptide confirmed the validity and accuracy of the X-ray structure. A picobody was constructed in which this N terminal portion was deleted and it was observed that function was still preserved (preliminary evidence suggests stronger binding as well). The smaller size of this molecule may make it a better vehicle for diagnostic and therapeutic applications.

The present disclosure describes a shorter nanobody which is now termed a “picobody” owing to its smaller size (8 fewer N-terminal residues). Disclosed embodiments include a picobody, as well as compositions and uses thereof.

The examples provide picobodies against alpha synuclein. However, a similar approach may be used in manufacturing picobodies against other proteins. And the number of N-terminal residues that are truncated may range from 1 to 20.

The present disclosure provides picobodies, and compositions including picobodies. In embodiments, the picobody may bind to alpha synuclein.

In embodiments, disclosed picobodies may include truncated nanobodies, for example, N-truncated nanobodies.

The present disclosure provides compositions, such as compositions including at least one picobody, for example alpha synuclein. In embodiments, the composition may include a pharmaceutically-acceptable carrier.

In embodiments, a patient suspected of having a neurodegenerative disease characterized by the abnormal accumulation of aggregates of alpha synuclein protein in neurons may display executive dysfunction, behavioral disturbances, or combinations thereof. Disclosed embodiments include treatment of a neurodegenerative disease with a picobody as described herein, for example an alpha synuclein picobody.

In embodiments, a patient with a neurodegenerative disease is diagnosed.

Disclosed embodiments include methods and compositions for reducing or preventing alpha synuclein aggregation.

Disclosed methods include diagnosis and treatments of synucleinopathies.

Subjects suitable for the disclosed methods and treatments may include for example, mammals, such as humans or animals.

Disclosed compositions include picobodies, for example picobodies that bind to alpha synuclein.

Disclosed methods include compositions including picobodies, for example, picobodies that bind to alpha synuclein.

Disclosed embodiments include kits for use in methods disclosed herein.

Disclosed embodiments include methods for predicting which antibody protein residues are relevant for binding alpha synuclein.

Disclosed embodiments include methods for producing picobodies.

Aspects of the present disclosure are directed to a picobody that includes an N-terminal truncation of an alpha synuclein nanobody. The picobody is configured to bind to an alpha synuclein, and the picobody is configured to inhibit aggregation of the alpha synuclein.

In some embodiments, the picobody includes an N-terminal truncation of an alpha synuclein nanobody.

In some embodiments, the N-terminal truncation is by 8 amino acid residues.

In some embodiments, the N-terminal truncation is by 1, 2, 3, 4, 5, 6, 7, 8, 9, 10, 11 or 12 amino acid residues.

In some embodiments, the binding to alpha synuclein is specific.

In some embodiments, the picobody includes a complementarity-determining region 3 (CDR3).

In some embodiments, the picobody binds to monomers and oligomers of alpha synuclein.

In some embodiments, the picobody has a molecular weight of about 12 to 15 Kda.

Another aspect of the present disclosure is directed to a pharmaceutical composition that includes the picobody disclosed above.

In some embodiments, the pharmaceutical composition further includes a pharmaceutically acceptable carrier.

Another aspect of the present disclosure is directed to a method of treating a neurodegenerative disease characterized by the accumulation of aggregates of alpha synuclein. The method includes administering to a patient in need thereof an effective amount of a picobody that is configured to bind to an alpha synuclein, and inhibit aggregation of the alpha synuclein.

In some embodiments, the picobody includes an N-terminal truncation of an alpha synuclein nanobody.

In some embodiments, the N-terminal truncation is by 8 amino acid residues.

In some embodiments, the N-terminal truncation is by 1, 2, 3, 4, 5, 6, 7, 8, 9, 10, 11 or 12 amino acid residues.

In some embodiments, the binding to alpha synuclein is specific.

In some embodiments, the picobody binds to monomers and oligomers of alpha synuclein.

In some embodiments, the picobody has a molecular weight of about 12 to 15 Kda.

In some embodiments, the neurodegenerative disease is Alzheimer's disease.

In some embodiments, the neurodegenerative disease is Parkinson's disease.

Another aspect of the present disclosure is directed to a method of diagnosing a neurodegenerative disease characterized by the accumulation of aggregates of alpha synuclein. The method includes detecting the presence of an alpha synuclein in a patient sample using a picobody that includes an N-terminal truncation of an alpha synuclein nanobody that is configured to bind to the alpha synuclein.

In some embodiments, the patient sample may be whole blood, serum, plasma, urine, tissue biopsies, saliva, sputum, cerebrospinal fluid (CSF), nasal swabs, tissue fluids, or biopsied cells or tissues.

In some embodiments, the picobody includes an N-terminal truncation of an alpha synuclein nanobody.

In some embodiments, the N-terminal truncation is by 8 amino acid residues.

In some embodiments, the N-terminal truncation is by 1, 2, 3, 4, 5, 6, 7, 8, 9, 10, 11 or 12 amino acid residues.

In some embodiments, the binding to alpha synuclein is specific.

In some embodiments, the picobody binds to monomers and oligomers of alpha synuclein.

In some embodiments, the picobody has a molecular weight of about 12 to 15 Kda.

In some embodiments, the neurodegenerative disease is Alzheimer's disease or Parkinson's disease.

Another aspect of the present disclosure is directed to an alpha synuclein picobody.

In some embodiments, the picobody includes an N-terminal truncation of an alpha synuclein nanobody.

Another aspect of the present disclosure is directed to a composition that includes an alpha synuclein picobody.

In some embodiments, the picobody includes an N-terminal truncation of an alpha synuclein nanobody.

Another aspect of the present disclosure is directed to a method of treating a patient with a neurodegenerative disorder. The method includes administering to the patient a composition that includes an alpha synuclein picobody.

In some embodiments, the picobody includes an N-terminal truncation of an alpha synuclein nanobody.

### EXAMPLES

### Example 1—Picobody Design and Production

Recently, the El-Agnaf laboratory at QBRI developed and studied a nanobody targeting alpha synuclein (NbM01) which has high affinity to both monomers and oligomers of alpha synuclein. This nanobody was shown to bind and also prevent aggregation of alpha synuclein thus making it a diagnostic and potentially therapeutic nanobody. The Kolatkar laboratory subsequently crystallized an NbM01-alpha synuclein complex and studied its three-dimensional structure using X-ray crystallography. Using a combination of home source and synchrotron X-rays (Brookhaven Lab), the Kolatkar group obtained a high resolution (1.25 angstroms) structure which allowed them to find atomic level detail of the complex. The complex revealed that the alpha synuclein is bound to a portion of nanobody which previous proteins or peptides didn't bind to. This portion was away from the central portion of the complementarity-determining region 3 (CDR3) relative to previously-determined structures and in addition the alpha synuclein peptide displaced the N-terminal end of the nanobody. Alanine scanning of the alpha synuclein peptide confirmed the validity and accuracy of the X-ray structure.

### Example 2—Treatment of PD

A Parkinson's disease (PD) patient is treated with an alpha synuclein picobody to prevent disease progression. The alpha synuclein picobody is formulated in a pharmaceutically acceptable carrier.

### Example 3—Treatment of AD

An Alzheimer's disease (AD) patient is treated with an alpha synuclein picobody to prevent disease progression. The alpha synuclein picobody is formulated in a pharmaceutically acceptable carrier.

“A” and “an” are used herein to refer to one or to more than one (i.e., to at least one) of the grammatical object of the article. By way of example, “an element” means one element or more than one element.

“Comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

“In vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

“Or” as used herein should be understood to mean “and/or”, unless the context clearly indicates otherwise.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

The term “treatment” may refer to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

“Reducing,” “suppressing” and “inhibiting” have their commonly understood meaning of lessening or decreasing.

In the context of antibody binding, “specific” refers to the highly selective and precise recognition and interaction between the antibody and its intended target, in this case, alpha synuclein. It means that the antibody demonstrates a strong affinity and preference for binding to alpha synuclein, distinguishing it from other proteins or molecules present in the environment.

As used herein, “pharmaceutically acceptable salts” refers to an ionizable therapeutic agent that has been combined with a counter-ion to form a neutral complex. Lists of suitable salts are found, for example, in Remington's Pharmaceutical Sciences, 17th ed., Mack Publishing Company, Easton, Pa., 1985, p. 1418, Journal of Pharmaceutical Science, 66, 2 (1977), and “Handbook of Pharmaceutical Salts: Properties, Selection, and Use” (P. Henrich Stahl & Camille G. Wermuth (Eds.), VHCA & Wiley-VCH, 2002).

The terms “pharmaceutical,” “pharmaceutically acceptable” and “therapeutically acceptable” may refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. These substances do not interfere with the effectiveness or the biological activity of the active ingredients.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal, and oral administration. “Pharmaceutically acceptable carrier” also includes agents for preparation of aqueous dispersions and sterile powders for injection or dispersions.

Pharmaceutically acceptable carriers, adjuvants and vehicles that may be used in the pharmaceutical compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present disclosure include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product. Also, see, for example, Ansel's Pharmaceutical Dosage Forms and Drug Delivery Systems, Wolters Kluwer Health (11th ed. 2018).

In some embodiments, any one of the compounds and therapeutic agents disclosed herein are administered orally. Compositions of the present disclosure suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

The pharmaceutical compositions of the present disclosure may be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present disclosure is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Examples of useful dermatological compositions which can be used to deliver the compounds to the skin are known in the art; see for example, Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The compounds and therapeutic agents of the present disclosure may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present disclosure provides an implantable drug release device impregnated with or containing a compound or a therapeutic agent, or a composition comprising a compound of the present disclosure or a therapeutic agent, such that said compound or therapeutic agent is released from said device and is therapeutically active.

Dosages and Regimens

In the pharmaceutical compositions of the present disclosure, a picobody or a pharmaceutically acceptable salts is present in an effective amount (e.g., a therapeutically effective amount).

Effective doses may vary, depending on the diseases treated, the severity of the disease, the route of administration, the sex, age and general health condition of the subject, excipient usage, and the possibility of co-usage with other therapeutic treatments such as use of other agents and the judgment of the treating physician.

In some embodiments, an effective amount of a picobody or pharmaceutically acceptable salt thereof can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a picobody or pharmaceutically acceptable salt thereof is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

In some embodiments, provided herein are packaged dosage forms, comprising a container holding a therapeutically effective amount of a picobody or pharmaceutically acceptable salt thereof, and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include 2-(2-cyanophenylthio)benzoic acid or an analog or derivative or a pharmaceutically acceptable salt thereof (and/or one or more of the other listed TA analogs or pharmaceutically acceptable salts thereof). The dosage forms provided herein may need storage lasting up to, or longer than, three months; in some cases up to, or longer than one year. The containers may be in any form suitable to contain the contents—for example, a bag, a bottle, or a box.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

The terms “effective,” “effective amount” or “therapeutically effective amount” refer to an amount, i.e., a dosage, of therapeutic agent administered to a subject (e.g., a mammalian subject, i.e., a human subject), either as a single dose or as part of a series of doses, which is effective to produce a desired therapeutic effect (e.g., effective for influencing, reducing or inhibiting the activity of or preventing activation of a kinase, or effective at bringing about a desired in vivo effect in an animal, preferably, a human, such as reduction in intraocular pressure).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A picobody,
wherein the picobody comprises an N-terminal truncation of an alpha synuclein nanobody,
wherein the picobody is configured to bind to an alpha synuclein, and
wherein the picobody is configured to inhibit aggregation of the alpha synuclein.

2. The picobody of claim 1, wherein the N-terminal truncation is by 8 amino acid residues.

3. The picobody of claim 1, wherein the binding to alpha synuclein is specific.

4. The picobody of claim 1, wherein the picobody comprises a complementarity-determining region 3 (CDR3).

5. The picobody of claim 1, wherein the picobody binds to monomers and oligomers of alpha synuclein.

6. The picobody of claim 1, wherein the picobody has a molecular weight of about 12 to 15 Kda.

7. A pharmaceutical composition comprising the picobody of claim 1.

8. A method of treating a neurodegenerative disease characterized by the accumulation of aggregates of alpha synuclein, the method comprising:
administering to a patient in need thereof an effective amount of a picobody,
wherein the picobody comprises an N-terminal truncation of an alpha synuclein nanobody that is configured to bind to an alpha synuclein and inhibit aggregation of the alpha synuclein.

9. The method of claim 8, wherein the N-terminal truncation is by 8 amino acid residues.

10. The method of claim 8, wherein the binding to alpha synuclein is specific.

11. The method of claim 8, wherein the picobody binds to monomers and oligomers of alpha synuclein.

12. The method of claim 8, wherein the picobody has a molecular weight of about 12 to 15 Kda.

13. The method of claim 8, wherein the neurodegenerative disease is Alzheimer's disease.

14. The method of claim 8, wherein the neurodegenerative disease is Parkinson's disease.

15. A method of diagnosing a neurodegenerative disease characterized by the accumulation of aggregates of alpha synuclein, the method comprising:
detecting the presence of an alpha synuclein in a patient sample using a picobody,
wherein the picobody comprises an N-terminal truncation of an alpha synuclein nanobody that is configured to bind to the alpha synuclein.

16. The method of claim 15, wherein the N-terminal truncation is by 8 amino acid residues.

17. The method of claim 15, wherein the binding to alpha synuclein is specific.

18. The method of claim 15, wherein the picobody binds to monomers and oligomers of alpha synuclein.

19. The method of claim 15, wherein the picobody has a molecular weight of about 12 to 15 Kda.

20. The method of claim 15, wherein the neurodegenerative disease is Alzheimer's disease or Parkinson's disease.
